(19) United States
(12) Patent Application Publication
(10) Pub. No.: US 2013/0225987 A1
(43) Pub. Date: Aug. 29, 2013

(54) DEDICATED BREAST IMAGING WITH IMPROVED GAMMA-RAY COLLECTION

(71) Applicant: Stephen Yarnall, Poway, CA (US)

(72) Inventor: Stephen Yarnall, Poway, CA (US)

(21) Appl. No.: 13/825,314

(22) PCT Filed: Sep. 19, 2011
(86) PCT No.: PCT/US11/52218

§ 371 Date: May 4, 2013

Related U.S. Application Data
(60) Provisional application No. 61/384,488, filed on Sep. 20, 2010.

(51) Int. Cl. A61B 6/02

(52) U.S. Cl. CPC A61B 6/025
USPC 600/426; 600/425

(57) ABSTRACT

A dedicated positron-emission tomography (“PET”) imaging device adapted to perform medical imaging procedures is described. The device includes at least two detector envelopes, each detector envelope adapted to conform to, contact, and stabilize an anatomical region of a patient, at least two gamma cameras, each coupled to an associated detector envelope, and multiple mechanical stages coupled to each of the gamma cameras, each mechanical stage adapted to provide an axis of movement for each of the cameras. In addition, a method of performing an imaging scan of an anatomical region is described. The method includes performing a pre-scan to determine a distribution of a radiotracer, determining a set of scan parameters based at least partly on results of the pre-scan, performing a comprehensive scan based at least partly on the set of scan parameters, and constructing an image based at least partly on the comprehensive scan.

### CROSS-REFERENCE TO RELATED APPLICATIONS

This Application claims benefit of an earlier-filed U.S. Provisional Patent Application entitled “Improving Gamma-ray Collection in a Dedicated Breast Imager,” filed on Sep. 20, 2010, and having Ser. No. 61/384,488. U.S. Provisional Patent Application 61/384,488 is incorporated herein by reference.

### BACKGROUND

Nuclear-emission imaging is commonly used to measure and display the physical distribution of radioactive tracers within a patient. The distribution of tracers can be used to indicate medically-important characteristics such as biochemical processes (e.g. glucose metabolism), flow through anatomical pathways (e.g. blood, lymph, etc.), and structures (e.g. blood vessels, lymph nodes, etc.). The radiotracer may selectively concentrate sufficiently to indicate specific characteristics, but the radiotracer may also be widely distributed making the characteristic difficult to distinguish, as is typical with any measurement that relies on the ratio of signal to noise for contrast.

Generalized nuclear emission imaging systems must be adaptable for various imaging tasks and therefore are not optimized for any specific imaging task(s). Application-specific or “dedicated” imaging systems that are intended to serve only limited purposes (e.g. brain or breast imaging) can have a higher degree of optimization for their specific application. Increasingly, molecular breast imaging using single photon emitting (e.g. sestamibi) and positron emitting (e.g. FDG—an analogue of glucose) radiotracers together with scintigraphy cameras or positron-emission tomography (“PET”) systems are being used to detect primary breast cancer. These systems utilize detectors and breast stabilization means that routinely allow the detection of small (<1 cm) breast tumors. These systems are configured to detect local breast cancer (i.e. localized to the breast).

One problem relating specifically to the efficient collection of photons in a dedicated breast imager includes accessing the deep breast tissue near the chest wall and axilla (noting that the diagnostic value of a breast imaging procedure that excludes a portion of breast is zero for the excluded portion). While general purpose imaging systems (e.g. whole body PET scanners) thoroughly survey these difficult to reach regions, they lack the image fidelity and the collection efficiency needed to characterize the common small and vague abnormalities (e.g. tumors) within the breast and are therefore unsuitable for breast imaging. Compact extremity, or organ-specific imagers which may otherwise be suitable for breast imaging, are unable to examine posterior breast tissues due to the physical barriers and dead space created by their orthogonal and orthogonal-cylindrical detector envelops, which abut the rib cage and the pectoral muscles. PET systems are further limited in their ability to examine posterior tissues by the loss of sensitivity at the periphery of their detectors, which results from fewer available coincident lines of response for acquiring image data.

Current imaging techniques are limited by collection efficiencies. A sufficient number of gamma-rays must be collected such that a radiotracer “signal” from the characteristic can be distinguished from “noise” produced by the widely distributed radiotracer. Current methods to increase the collection of gamma-rays include increasing the strength of the radiotracer dose administered to the patient, the length of the acquisition period, and the size and sensitivity of the detector. There are, however, undesirable tradeoffs with each of these methods.

Increasing the radiotracer dose increases the potential for radiation-induced tissue damage (e.g. carcinogenic changes) to the patient or healthcare providers. Higher doses of radioactive tracers increase the risk of direct cellular damage as well as damage to DNA strands, which can induce carcinogenesis. Less-efficient photon-collecting detector systems require higher radioisotope dosing, which increases the potential for cellular damage.

Increasing the image-acquisition time in order to achieve sufficient contrast increases procedure costs and any effects of radiotracer dynamics (e.g. pharmacodynamics, pharmacokinetics, motion blurring from patient movement) on the image. Photon collection time directly effects procedure time and longer procedures can tire the patient, resulting in patient movement, which may blur the resulting image. In addition, radiotracer distribution can change during the procedure, confounding the diagnosis. For example, during a long FDG-PET procedure, the urinary bladder may fill with radiotracer-rich urine, changing the distribution of background counts, which changes the image contrast.

Increasing the size of the gamma-ray detectors (including materials and/or related electronics) increases the costs of manufacturing the imaging system. These materials commonly make up the majority of the materials costs for constructing an imaging system. Under-utilized detector materials result in a decrease in the cost-specific photon-collection efficiency of a dedicated breast imager, for example, when a large detector is used to survey a small breast.

Thus, there exists a need for a way to perform nuclear-emission imaging that is dedicated to a specific body part, has a high collection efficiency that limits radiotracer dosage, allows high image quality that may be manufactured and operated at low cost.

### BRIEF SUMMARY

Some embodiments of the present invention provide a way to optimize collection of gamma-rays being from a body part during a molecular imaging nuclear medical procedure. Some embodiments provide an apparatus that may be used to position the detector(s) in very close proximity to a body part being evaluated in order to increase the rate of gamma-ray collection. The apparatus may also be used to move the detector(s) in relation to the body part such that a small detector may be used to cover a larger body part, and to selectively spend more time collecting gamma-rays from a particular area or areas than another area or other areas. The apparatus may also be used to position the detectors in close proximity to reach difficult regions of the anatomy (e.g. posterior breast tissue, axillary nodal tissue, etc.).

The apparatus may include a gamma camera positioned within an enclosure. The enclosure may be used to stabilize a body part with respect to the space coordinates of the gamma camera by compression or surface contact. Surfaces of the enclosure may be curved to conform to the surrounding anatomy for providing full access to the body part, thus enabling a comprehensive survey. The enclosure may also be contoured to conform to the body part to eliminate gaps between the body part and the enclosure, maintaining close proximity between the body part and the gamma camera. The gamma camera may have a limited field of view in comparison to the size of the body part, allowing savings on materials and construction costs. The gamma camera may be repositioned within the enclosure to complete a survey of the entire body part. The apparatus may include more than one gamma camera, a gamma camera ring or a partial ring gamma camera, all of which may be used to collect coincident gamma rays as in a PET system.

Mechanical stages may be used to move the gamma camera(s) within the enclosure along at least two axes, thus allowing the camera(s) to be optimally positioned with respect to the body part for efficient photon collection. The enclosure may provide bearing surfaces needed to stabilize the camera movement on at least the surface of the enclosure facing the body part, to eliminate unessential materials or gaps and insure that a minimum distance can be maintained between the gamma camera and the body part to reduce photon attenuation and increase photon capture. The photonic elements of the gamma camera may be arranged to prioritize the collection of gamma rays from one area over another.

A method of some embodiments may be used to position the detector(s) in relation to the body part such that a sufficient number of gamma-rays to produce an image with adequate contrast are collected in a minimum of time for a detector of a particular size.

The method may provide a way of utilizing the apparatus to optimally plan and complete the radio-anatomical survey, by selectively positioning and moving of the gamma camera within the enclosure based on one or more relevant factors. Such factors may include, for instance, an outline of the body part such that no time is wasted acquiring photons from outside the body part, a concentration of photons collected from any volume within the body part, other information regarding radiotracer distribution acquired during a short “pre-scan”, a sensitivity profile of the gamma camera(s), the number of photons needed to depict a statistically-valid distribution of radio-tracer within the body part, historical information from previous image data-sets, and/or patient data.

Some embodiments provide a dedicated positron-emission tomography PET imaging device adapted to perform medical imaging procedures. The device may include at least two detector envelopes, each detector envelope adapted to conform to, contact, and stabilize, with respect to the space coordinates of the detector envelope, an anatomical region of a patient under evaluation. In addition, the device may include at least two gamma cameras, each gamma camera coupled to an associated detector envelope from the at least two detector envelopes.

The device may also include multiple mechanical stages coupled to each of the gamma cameras, each mechanical stage adapted to provide an axis of movement for each of the gamma cameras. Each detector envelope of the imaging device may be adapted to provide a set of bearing surfaces for stabilizing each gamma camera. The cameras and mechanical stages of the imaging device may be adapted to allow the camera to be moved along an arc. In some embodiments, the anatomical region of the dedicated imaging device is a breast. Photonic elements of the gamma cameras of the imaging device may be asymmetrically arranged to allow collection of gamma rays from the posterior breast. Each detector envelope of the imaging device may be contoured to conform to a rib cage of the patient and to accommodate pectoral muscles of the patient.

Some embodiments provide a PET detector envelope adapted to sense gamma ray emissions from an anatomical region of a patient under evaluation. The gamma camera may be adapted to detect gamma radiation emitting radioisotopes. In addition, the detector may include a set of mechanical stages coupled to the gamma camera, each mechanical stage adapted to provide an axis of movement for the gamma camera within the detector envelope. The detector envelope may be adapted to conform to the anatomical region and a gamma camera coupled to the detector envelope.

In some embodiments, the camera and set of mechanical stages of the detector are adapted to allow the camera to be moved along an arc. The set of mechanical stages of the detector may include at least two mechanical stages. The envelope of the detector may be further adapted to provide a set of bearing surfaces for stabilizing each gamma camera. In some embodiments, the anatomical region is a breast. Photonic elements of the gamma cameras of the detector may be asymmetrically arranged to allow collection of gamma rays from the posterior breast.

Some embodiments provide a method of performing a nuclear-emission imaging scan of an anatomical region of a patient under evaluation. The method may perform a pre-scan to determine a distribution of a radiotracer over a pre-scan region comprising the anatomical region. The pre-scan may maneuver a set of gamma camera in a survey pattern over the pre-scan region and determine a set of discontinuities and gradients in the distribution of the radiotracer. In addition, the method may determine a set of scan parameters based at least partly on results of the pre-scan, perform a comprehensive scan of the anatomical region based at least partly on the set of scan parameters, and construct an image based at least partly on the comprehensive scan.

In some embodiments, the anatomical region is smaller than the pre-scan region. The set of camera may be arranged in an asymmetrical manner. Determining the set of scan parameters may include determining a path for each gamma camera and determining a rate of movement for each gamma camera. Some embodiments may also adjust acquisition parameters while performing the comprehensive scan, the adjustment based at least partly on the results of the pre-scan. The acquisition parameters may include line of response acceptance angles, energy spectrum, and coincidence timing window. The anatomical region scanned by the method is a breast in some embodiments.

The preceding Summary is intended to serve as a brief introduction to some embodiments of the invention. The summary is not meant to be an introduction or overview of all inventive subject matter disclosed herein. The Detailed Description that follows and the Drawings (or “FIGs.”) that are referred to in the Detailed Description further describe the embodiments described in the Summary as well as other embodiments. Accordingly, to understand all the embodiments described by this document, a full review of the Summary, Detailed Description and the Drawings is needed. Moreover, the claimed subject matter is not to be limited by the illustrative details in the Summary, Detailed Description and the Drawings, but rather is to be defined by the appended claims, because the claimed subject matter may be embodied in other specific forms without departing from the spirit of the invention.

### BRIEF DESCRIPTION OF THE DRAWINGS

The novel features of the invention are set forth in the appended claims. However, for purpose of explanation, several embodiments of the invention are set forth in the following drawings.

FIG. 1A illustrates a perspective view of a breast imaging device, in use, according to some embodiments of the invention;

FIG. 1B illustrates a perspective view highlighting body-conforming curved detectors of some embodiments of the breast imaging device of FIG. 1A;

FIG. 2 illustrates the increased coverage, compared with an orthogonal detector, of a breast image generated by some embodiments of breast imaging device of FIG. 1A;

FIG. 3 illustrates a side view of a gamma camera housed in a curved detector envelope of some embodiments of the breast imaging device of FIG. 1A;

FIG. 4 illustrates a top view of a moveable gamma camera-to-detector enclosure interface using movable bearing surfaces of some embodiments of the breast imaging device of FIG. 1A;

FIG. 5 illustrates a flow chart showing a process used for systematic imaging using a moveable gamma camera according to some embodiments of the invention; and

FIG. 6 conceptually illustrates a computer system 600 with which some embodiments of the invention may be implemented.

### DETAILED DESCRIPTION

In the following detailed description of the invention, numerous details, examples, and embodiments of the invention are set forth and described. However, it will be clear and apparent to one skilled in the art that the invention is not limited to the embodiments set forth and that the invention may be practiced without some of the specific details and examples discussed.

Several more detailed embodiments of the invention are described in the sections below. Section I provides a conceptual description of the system and devices used by some embodiments to perform body part imaging. Section II then describes a process of some embodiments that may be implemented to perform a body part scan. Lastly, Section III describes a computer system which may at least partially implement some embodiments of the invention.

Although various examples of body parts may be provided below (e.g., a breast), one of ordinary skill in the art will recognize that other specific body parts may be used with different embodiments of the invention without departing from the spirit of the invention. In addition, although reference may be made below to various specific hardware and/or software elements, one of ordinary skill in the art will recognize that different embodiments may implement various components and/or functionality in various different ways. For example, different embodiments may use differently-shaped detectors.

### I. System

As used herein, the term “detector” may include at least a gamma camera and a capability to stabilize a body part with respect to the camera. A gamma camera (also called a “scintillation camera” or “Anger camera”), is a device that may be used to detect gamma radiation emitting radioisotopes, a technique known as scintigraphy. The surfaces of such a gamma camera may be used to stabilize the body part. Alternatively, a “detector envelope” may be situated between the gamma camera and the body part for stabilization (e.g. when the gamma camera is moveable with respect to the body part, when an intermediate surface is desirable, etc.).

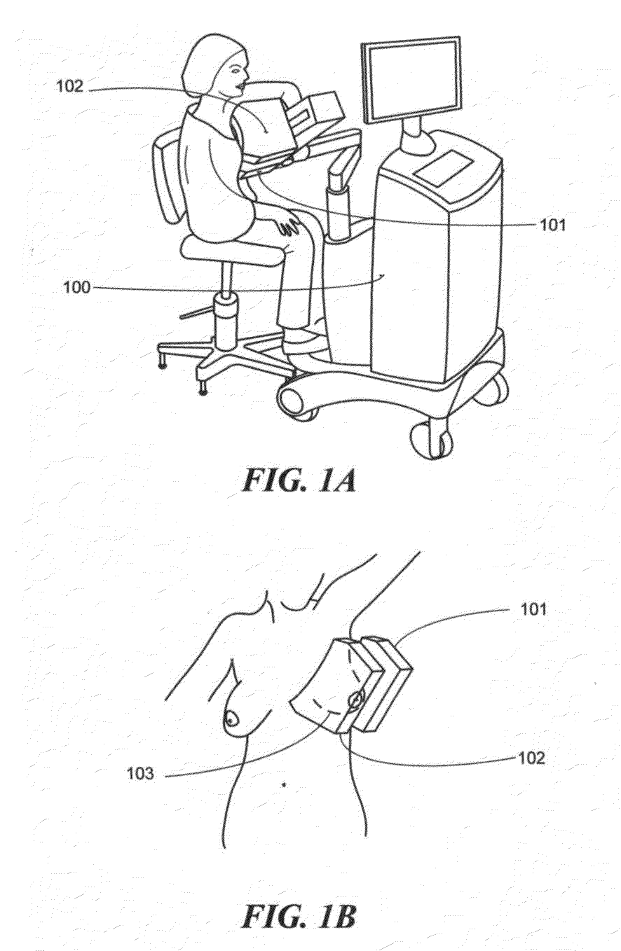

FIG. 1A illustrates a perspective view of a dedicated PET breast imaging system 100, in use, according to some embodiments of the invention. Specifically, this FIG. shows the device as used during a typical patient imaging procedure. As shown, the device includes multiple detectors 101 and 102 for sensing gamma-ray emissions. The detectors may be positioned in various ways (e.g., by moving the detectors along an “arm” of the device, by turning the detectors on a pivot, by clamping the detectors in place, etc.) during use. In this way, the detectors may be placed at an appropriate location to scan a particular body part (e.g., a breast).

FIG. 1B illustrates a perspective view highlighting body-conforming curved detectors of some embodiments of the breast imaging device 100. Specifically, this figure shows the placement of the adaptors allowed by the body-conforming shaped edge 103. As shown, the curved shape of the edge conforms, for example, to the rib cage and accommodates pectoral muscles, providing access to image the posterior breast region. The characteristics of the curve may be adjusted for each of the opposing detectors to fit the body shape or other characteristics of the patient and may be positioned at a desired orientation for coupling the imaging system to the patient. In standard mammographic positioning, the medial-lateral-oblique orientation (“MLO”) shown in FIG. 1A is usually able to encompass more breast tissue for imaging than other orientations.

Some embodiments may provide “quick-swap” detector envelopes. Such envelopes may be made in different sizes (e.g., to fit a large patient, a small patient, etc.) and/or shapes (e.g., to fit various body parts such as the breast, axilla, groin, etc.). Various shapes may also be used to fit differently-sized patients (i.e., the characteristics of the curve may be changed for different envelopes to fit the characteristics of differently-sized patients). For instance, a larger person may have a more convex shaped rib cage while a smaller person may have a flatter or more concave shaped rib cage.

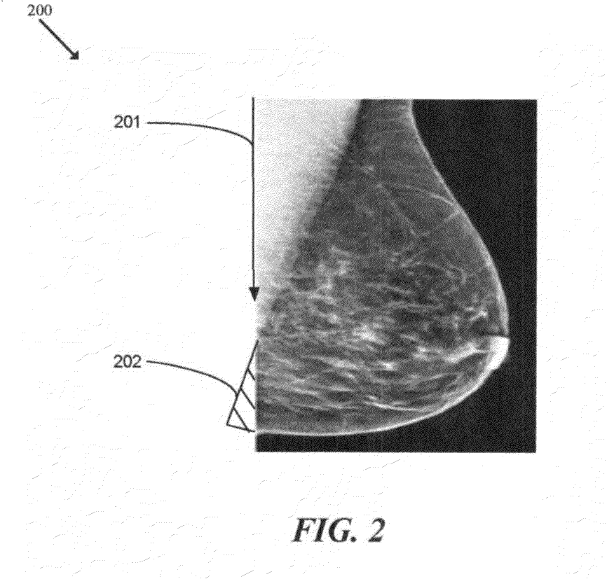

FIG. 2 illustrates the increased coverage, compared with an orthogonal detector, of a breast image generated by some embodiments of breast imaging device of FIG. 1A. Specifically, this figure shows an additional area of the breast that may be scanned using a dedicated system. As shown, due to the straight (orthogonal) edge 201 of some imaging systems, some areas of the breast 202 that are included in a dedicated system cannot be encompassed. With a properly sized and/or shaped detector that may be able to accommodate the curvature of the rib cage and pectoral muscles with the detector envelope, nearly, if not all of the patient's breast can be encompassed and imaged in a single MLO positioning. Provided the imaging system has adequately-isotropic resolution and contrast, a single MLO image acquisition may provide a comprehensive breast examination, eliminating the need for additional alternate orientation acquisitions and shortening the imaging procedure.

The gamma camera may conform closely to the curved detector envelope to take advantage of the increased access for imaging. Although typical gamma cameras are usually configured to be orthogonal (e.g., single-photon emission computed tomography (“SPECT”) gamma cameras, cylindrical-orthogonal (“PET”) gamma cameras, or planar-orthogonal-opposing (positron emission mammography (“PEM”) gamma cameras), various ways for arranging non-orthogonal (curved) configurations, such as asymmetrical scintillators with light guides and optical fibers may be used to achieve close gamma camera-to-chest-wall conformance. Alternatively, the gamma camera may not conform closely to any shape of the envelope (e.g., the camera may be sized to fit within the envelope, or a portion of the envelope, without necessarily conforming to the shape of the envelope).

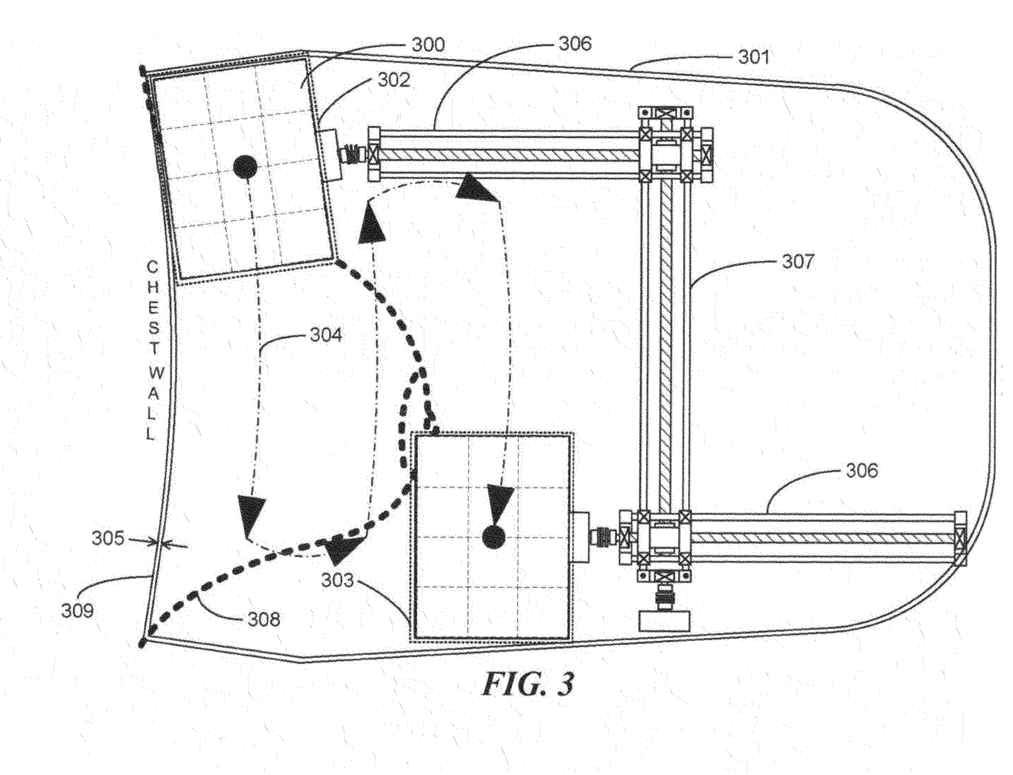

FIG. 3 illustrates a side view of a gamma camera 300 housed in a curved detector envelope 301 of some embodiments of the breast imaging device of FIG. 1A. Specifically, this figure shows the movement of the camera within the detector envelope used as an alternative to shaping the gamma camera into a non-orthogonal orientation. As shown, an orthogonal gamma camera 300 may be moved from a first position 302 to a second position 303, along a path 304 that conforms to the curved portion 305 of the detector envelope 301, using mechanical stages 306 and 307. The detector envelope may be in intimate contact with the breast 308 and may then be used to correlate the space coordinates of the moving gamma camera with respect to the breast.

By moving the gamma camera, a larger field of view may be surveyed, affording cost advantages compared to detector with a stationary “full-field” gamma camera. Various body parts and fields-of-view may be accommodated by exchanging the detector envelope 301 including the curved surface adjacent to the chest wall 309 for a differently shaped and/or differently sized detector envelope, and by re-programming the mechanical stages 306 and 307 that control the field of view. The detector path 304 may be pre-programmed and/or actively controlled to vary the time spent collecting (or not collecting) photons in a given area within the maximum field of view afforded by the detector envelope 301. An orthogonal detector may be used to trace a contoured detector envelope, provided the arc-length subtended by the surface of the detector is sufficiently small in comparison to the contour of the detector envelope.

The posterior portion of the breast is noted to have an increased tendency for cancer, thus it is a critical region to survey. As shown in FIG. 3, the aspect ratio of the detector 300 is not uniform;

rather it is longer along the surface adjacent to the patient's chest wall 309. In this way, more detector material is available in a given time period for collecting gamma photons at the extreme edge of the field of view (i.e., the edge corresponding to the chest wall and the posterior breast). This configuration may be especially important in PET systems that have lower sensitivity at the outside perimeter of the detectors due to fewer possible coincident lines of response (“LORs”). This configuration is also important in collimated single-photon nuclear emission systems that may need to process many more photons at the edge of the detector near the chest wall, where radiation scatter from the thorax tends to overwhelm the desired radiation signal from the breast.

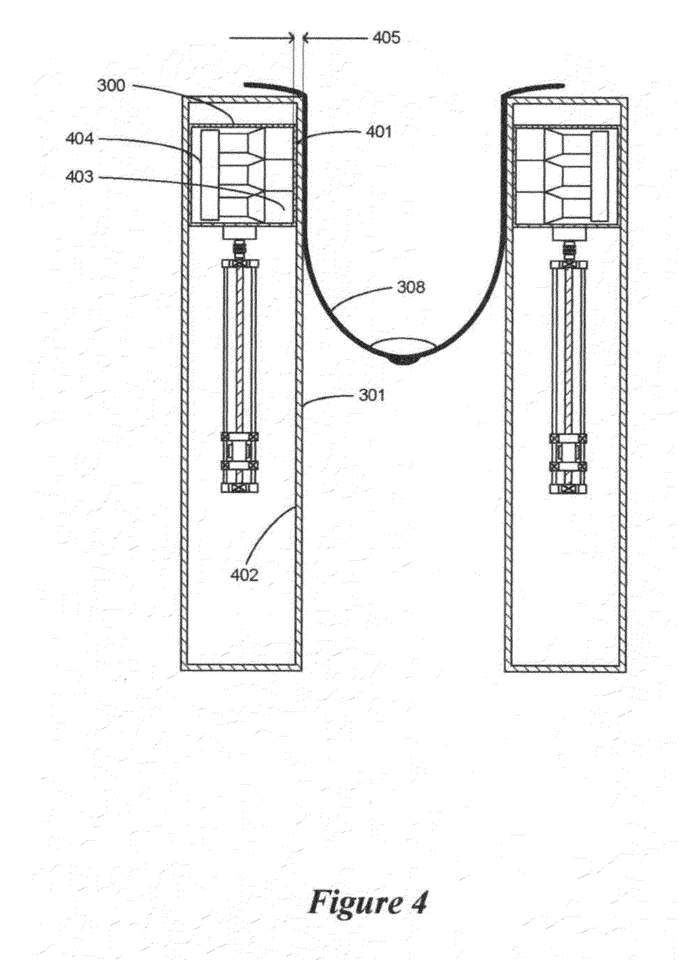

FIG. 4 illustrates a top view of a moveable gamma camera-to-detector enclosure interface using moveable bearing surfaces of some embodiments of the breast imaging device of FIG. 1A. Specifically, this figure shows the placement of the detectors relative to a breast 308 during imaging. As shown, the contact interface (or “contact surface”, or “bearing surface”) 401 of the gamma camera assembly 300 is coupled to the interior 402 of the detector envelope 301, which is in contract with the breast 308 under evaluation. Extraneous materials and air gaps may be eliminated by utilizing the contacting surface 401 of the gamma camera 300 and the detector envelope 402 in a slide-able bearing arrangement. In this way the scintillation stage 403 of the detector photonic elements 404 may be positioned as close as possible 405 to the breast 308 in order to maximize photon capture. In addition to the necessary radiation transparency, the bearing surfaces of the detector 401 and the detector enclosure 301 may have anti-friction and anti-wear properties able to endure repeated scanning

### II. Method of Operation

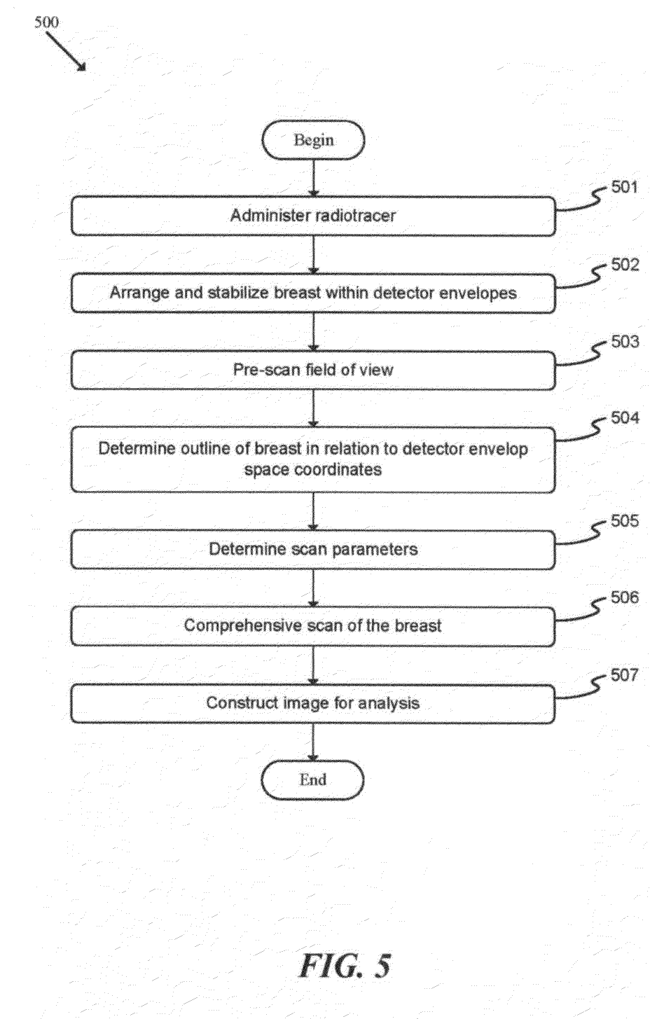

FIG. 5 illustrates a flow chart showing a process 500 used for systematic imaging using a moveable gamma camera according to some embodiments of the invention (e.g., the camera 300 described above in reference to FIG. 3). Such a process may be used to increase gamma-ray collection efficiency in a breast-dedicated ET imager with planar detectors during an FDG molecular imaging procedure. The process may begin when a patient evaluation procedure begins (e.g., when a patient begins a breast scan procedure). As shown, a radiotracer may be administered (at 501) in any appropriate way (e.g., orally, by injection, etc.). After the radiotracer has been administered to the patient 501 (e.g. injection, oral, inhaled) and the appropriate time has elapsed for radiotracer uptake, the breast 308 may be arranged and stabilized (at 502) within the detector envelopes (e.g., detectors 101 and 102 having envelope 301). Optionally, the thickness of the breast between the detectors (and/or other relevant information) may be determined and combined with other data (e.g. patient weight, height, dosimetry, breast size, breast density) which is then used to tailor the ensuing image acquisition processes.

A programmed pre-scan may then be performed (at 503). Such a pre-scan may scan the entire field of view within a particular detector envelope. In this way, the relevant portions of the envelope to be fully scanned may be determined. The pre-scan may roughly determine the distribution of the radiotracer. A minimum amount of time may be spent maneuvering the gamma cameras in a survey pattern such that the resulting image is able to be analyzed in order to determine (at 504) the space coordinates of the breast perimeter (extending away from the chest wall) between the two detector envelopes. Discontinuities and gradients in the image are analyzed to signal the outline of the breast using manual (e.g. visual, using a pointing device on the produced image, and/or other appropriate ways) or computational methods (e.g. that rely on Canny differential equations and/or other appropriate ways).

Next, the process may determine (at 505) scan parameters, which may include the gamma camera path and rate of movement. The camera path may be configured such that only the desired region of the envelope is scanned (e.g., only the area within the outline of the breast may be scanned). The rough distribution of radiotracer, the breast outline, the sensitivity profile of the gamma cameras, and other appropriate information (such as that gathered at 504) may be used to tailor the scan parameters, with the goal of providing statistically valid image contrast throughout the breast to enable a comprehensive reader interpretation or computer-aided detection (“CAD”) in a minimum acquisition period.

A comprehensive scan of the breast may then be performed (at 506). Various PET acquisition parameters such as line of response acceptance angles, energy spectrum, and coincidence timing window may be adjusted during the scan in specific areas based on pre-scan data to optimize collection efficiency, image resolution and contrast. In addition, the camera path and rate of movement may be adjusted during the comprehensive scan. For instance, the camera may be moved more slowly over an area with low levels of radiotracer.

During and after the scan, various and well-known methods for constructing (at 507) and processing the image dataset using PET technology are applied (e.g. random subtraction, attenuation correction, reconstruction, etc.) such that the image may be analyzed to ascertain the underlying medical condition.

### III. Computer System

Many of the processes and modules described above may be implemented as software processes that are specified as a set of instructions recorded on a computer readable storage medium (also referred to as “computer readable medium” or “machine readable medium”). When these instructions are executed by one or more computational element(s), such as processors (e.g., microprocessors) or other computational elements like Application-Specific ICs (“ASICs”), Field Programmable Gate Arrays (“FPGAs”), digital signal processors (“DSPs”), etc. the computational element(s) cause the computational element(s) to perform the actions indicated in the instructions. Computer is meant in its broadest sense, and can include any electronic device with at least one processor. Examples of computer readable media include, but are not limited to, CD-ROMs, flash drives, RAM chips, hard drives, EPROMs, “cloud” storage, etc. The computer readable media do not include carrier waves and/or electronic signals passing wirelessly or over a wired connection.

In this specification, the term “software” includes firmware residing in read-only memory or applications stored in magnetic storage which can be read into memory for processing by one or more processors. Also, in some embodiments, multiple software inventions can be implemented as sub-parts of a larger program while remaining distinct software inventions. In some embodiments, multiple software inventions can also be implemented as separate programs. Finally, any combination of separate programs that together implement a software invention described herein is within the scope of the invention. In some embodiments, the software programs when installed to operate on one or more computer systems define one or more specific machine implementations that execute and perform the operations of the software programs.

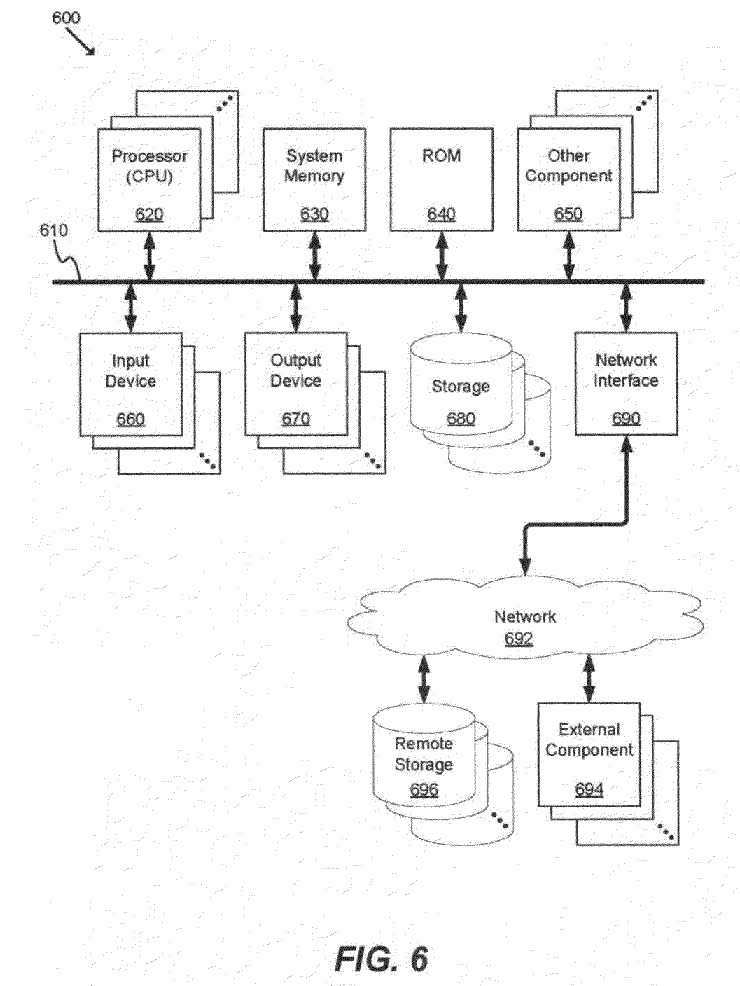

FIG. 6 conceptually illustrates a computer system 600 with which some embodiments of the invention may be implemented. For example, the system described above in reference to FIG. 1 may be at least partially implemented using computer system 600. As another example, the process described in reference to FIG. 5 may be at least partially implemented using sets of instructions that are run on the computer system.

Such a computer system may include various types of computer readable media and interfaces for various other types of computer readable media. As shown, computer system 600 may include a bus 610, at least one processing unit (e.g., a microprocessor or microcontroller) 620, a system memory 630, a read-only memory (ROM) 640, other components (e.g., a graphics processing unit) 650, input devices 660, output devices 670, permanent storage devices 680, and a network connection 690. The components of the computer system 600 are electronic devices that automatically perform operations based on digital and/or analog input signals.

One of ordinary skill in the art will recognize that the computer system 600 may be embodied in other specific forms without deviating from the spirit of the invention. For instance, the computer system may be implemented using various specific devices either alone or in combination. For example, a local PC may include the input devices 660 and output devices 670, while a remote PC may include the other devices 610-650 and 680, with the local PC connected to the remote PC through a network that the local PC accesses through its network connection 690 (where the remote PC is also connected to the network through a network connection).

The bus 610 represents all communication pathways that connect the numerous devices of the computer system 600. Such pathways may include wired, wireless, and/or optical communication pathways. For example, the input devices 660 and/or output devices 670 may be coupled to the system 600 using a wireless local area network (W-LAN) connection, Bluetooth®, or some other wireless connection protocol or system.

The bus 610 communicatively connects, for example, the processor 620 with the system memory 630, the ROM 640, and the permanent storage device 650. From these various memory units, the processor 620 retrieves instructions to execute and data to process in order to execute the processes of some embodiments. In some embodiments the processor includes an FPGA, an ASIC, or various other electronic components used to executing instructions.

The ROM 640 may store static data and instructions that are needed by the processor 620 and other modules of the computer system. The permanent storage device 650, on the other hand, may be a read-and-write memory device. This device may be a non-volatile memory unit that stores instructions and data even when the computer system 600 is off. Some embodiments of the invention may use a mass-storage device (such as a magnetic or optical disk and its corresponding disk drive) as the permanent storage device 650.

Other embodiments may use a removable storage device (such as a floppy disk, flash drive, or CD-ROM) as the permanent storage device. Like the permanent storage device 650, the system memory 630 may be a read-and-write memory device. However, unlike storage device 650, the system memory 630 may be a volatile read-and-write memory, such as a random access memory (RAM). The system memory may store some of the instructions and data that the processor needs at runtime. In some embodiments, the sets of instructions and/or data used to implement the invention's processes may be stored in the system memory 630, the permanent storage device 650, and/or the read-only memory 640.

The bus 610 may also connect to the input devices 660 and output devices 670. The input devices 660 may enable the user to communicate information and select commands to the computer system. The input devices may include alphanumeric keyboards, touchscreen inputs, and/or pointing devices (also called “cursor control devices”). The input devices may also include audio input devices (e.g., microphones, MIDI musical instruments, etc.) and video input devices (e.g., video cameras, still cameras, optical scanning devices, etc.). The output devices 670 may include printers, electronic display devices that display still or moving images, and electronic audio devices that play audio generated by the computer system. For instance, these display devices may display a GUI. The display devices may include devices such as cathode ray tubes (“CRT”), liquid crystal displays (“LCD”), plasma display panels (“PDP”), surface-conduction electron-emitter displays (alternatively referred to as a “surface electron display” or “SED”), etc. The audio devices may include a PC's sound card and speakers, a speaker on a cellular phone, a Bluetooth® earpiece, etc. Some or all of these output devices may be wirelessly or optically connected to the computer system.

Finally, as shown in FIG. 6, bus 610 may also couple computer 600 to a network interface 690 through which one or more networks 692 may be accessed. The computer 600 may connect to various external components 694 and remote storages 696 through the network 692. In this manner, the computer may be a part of a network of computers (such as a local area network (“LAN”), a wide area network (“WAN”), an Intranet, or a network of networks, such as the Internet. For example, the computer 600 may be coupled to a web server so that a web browser executing on the computer 600 can interact with the web server as a user interacts with a GUI that operates in the web browser.

As mentioned above, some embodiments may include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable blu-ray discs, ultra density optical discs, any other optical or magnetic media, and floppy disks. The computer-readable media may store a computer program that is executable by a device such as an electronics device, a microprocessor, a processor, a multi-processor (e.g., an IC with several processing units on it) and includes sets of instructions for performing various operations. The computer program excludes any wireless signals, wired download signals, and/or any other ephemeral signals.

Examples of hardware devices configured to store and execute sets of instructions include, but are not limited to, microprocessors, microcontrollers, DSPs, ASICs, FPGAs, programmable logic devices (“PLDs”), ROM, and RAM devices. Examples of computer programs or computer code include machine code, such as produced by a compiler, and files including higher-level code that are executed by a computer, an electronic component, or a microprocessor using an interpreter.

As used in this specification and any claims of this application, the terms “computer”, “server”, “processor”, and “memory” all refer to electronic or other technological devices. These terms exclude people or groups of people. For the purposes of this specification, the terms display or displaying mean displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and/or any other ephemeral signals.

It should be recognized by one of ordinary skill in the art that any or all of the components of computer system 600 may be used in conjunction with the invention. Moreover, one of ordinary skill in the art will appreciate that any other system configuration may also be used in conjunction with the invention or components of the invention.

Furthermore, while the examples shown illustrate many individual modules as separate blocks, one of ordinary skill in the art would recognize that some embodiments may combine these modules into a single functional block or element. One of ordinary skill in the art would also recognize that some embodiments may divide a particular module into multiple modules.

While the invention has been described with reference to numerous specific details, one of ordinary skill in the art will recognize that the invention can be embodied in other specific forms without departing from the spirit of the invention. For example, several embodiments were described above by reference to particular features and/or components (e.g., the detectors 101 and 102, the camera 300, etc.). However, one of ordinary skill in the art will realize that other embodiments might be implemented with other types of detectors with other types of features and components (e.g., differently shaped or differently sized detectors). As another example, several embodiments were described above by reference to a particular body part (e.g., the breast). However, one of ordinary skill in the art will recognize that other embodiments might be adapted to be dedicated to other body parts (or might be adapted for general rather than dedicated use). One of ordinary skill in the art would understand that the invention is not to be limited by the foregoing illustrative details, but rather is to be defined by the appended claims.

We claim:

1. A dedicated positron-emission tomography (“PET”) imaging device adapted to perform medical imaging procedures, the device comprising:
at least two detector envelopes, each detector envelope adapted to conform to, contact, and stabilize, with respect to the space coordinates of the detector envelopes, an anatomical region of a patient under evaluation; and
at least two gamma cameras, each gamma camera coupled to an associated detector envelope from the at least two detector envelopes.

2. The imaging device of claim 1 further comprising a plurality of mechanical stages coupled to each of the gamma cameras, each mechanical stage adapted to provide an axis of movement for each of the gamma cameras.

3. The imaging device of claim 2, wherein the cameras and mechanical stages are adapted to allow the camera to be moved along an arc.

4. The imaging device of claim 1, wherein each detector envelope is adapted to provide a set of bearing surfaces for stabilizing each gamma camera.

5. The imaging device of claim 1, wherein the anatomical region is a breast.

6. The imaging device of claim 5, wherein photonic elements of the gamma cameras are asymmetrically arranged to prioritize collection of gamma rays from the posterior breast.

7. The imaging device of claim 5, wherein each detector envelope is contoured to conform to a rib cage of the patient and to accommodate pectoral muscles of the patient.

8. A positron-emission tomography (“PET”) detector envelope adapted to sense gamma ray emissions from an anatomical region of a patient under evaluation, the detector envelope comprising:
a gamma camera positioned within the detector envelope, the gamma camera adapted to detect gamma radiation emitting radioisotopes; and
a set of mechanical stages coupled to the gamma camera, each mechanical stage adapted to provide an axis of movement for the gamma camera within the detector envelope,
wherein the detector envelope is adapted to conform to the anatomical region.

9. The detector envelope of claim 8, wherein the camera and set of mechanical stages are adapted to allow the camera to be moved along an arc.

10. The detector envelope of claim 8, wherein the set of mechanical stages comprises at least two mechanical stages.

11. The detector envelope of claim 8, wherein the envelope is further adapted to provide a set of bearing surfaces for stabilizing each gamma camera.

12. The detector envelope of claim 8, wherein the anatomical region is a breast.

13. The detector envelope of claim 12, wherein photonic elements of the gamma camera are asymmetrically arranged to prioritize collection of gamma rays from the posterior breast.

14. An automated method of performing a nuclear-emission imaging scan of an anatomical region of a patient under evaluation, the method comprising:
performing a pre-scan to determine a distribution of a radiotracer over a pre-scan region comprising the anatomical region, the pre-scan comprising:
maneuvering a set of gamma cameras in a survey pattern over the pre-scan region; and
determining a set of discontinuities and gradients in the distribution of the radiotracer;
determining a set of scan parameters based at least partly on results of the pre-scan;
performing a comprehensive scan of the anatomical region based at least partly on the set of scan parameters; and
constructing an image based at least partly on the comprehensive scan.

15. The automated method of claim 14, wherein the anatomical region is smaller than the pre-scan region.

16. The automated method of claim 14, wherein determining the set of scan parameters comprises:
determining a path for each gamma camera; and
determining a rate of movement for each gamma camera.

17. The automated method of claim 16, further comprising adjusting acquisition parameters for performing the comprehensive scan, the adjusting based at least partly on the results of the pre-scan.

18. The automated method of claim 17, wherein the acquisition parameters comprise at least one of line of response acceptance angles, energy spectrum, and coincidence timing window.

19. The automated method of claim 16, further comprising adjusting the rate of movement for each gamma camera during the comprehensive scan based at least partly on a number of photons acquired versus a number of photons needed to depict a statistically-valid distribution of the radiotracer within the anatomical region.

20. The automated method of claim 14, wherein the anatomical region is a breast.
